(19) United States
(12) Patent Application Publication
(10) Pub. No.: US 2006/0123560 A1
(43) Pub. Date: Jun. 15, 2006

(54) Textile treatment agent

(75) Inventors: Gerhard Johschker, Spiesen-Elversberg (DE); Sylvia Rastatter, Birkenau (DE)

Correspondence Address: HARNESS, DICKEY & PIERCE, P.L.C., P.O. BOX 828, BLOOMFIELD HILLS, MI, 48303, US

(73) Assignee: NANOGATE COATING SYSTEMS GMBH, Saarbrucken (DE)

(21) Appl. No.: 10/530,996

(22) PCT Filed: Oct. 17, 2003
(86) PCT No.: PCT/DE03/03476

§ 371 Date: Sep. 26, 2005

(30) Foreign Application Priority Data
Oct. 17, 2002 (DE) 102 48 583.6

(51) Int. Cl. A01H 5/02; D06F 35/00

(52) U.S. Cl. 8/147; 8/158

(57) ABSTRACT

The invention relates to a textile treatment agent for treating a textile that is to be brought into contact therewith, in particular, during a washing process. Said textile treatment agent contains at least one first textile treatment constituent and at least one other constituent. The invention provides that the first textile treatment constituent serves to form an inorganic structure on the textile surface, particularly on the surfaces of the textile fibers.

The present invention relates to the subject matter as described in the generic part of claim 1 and thus deals with the treatment of textiles.

There are a number of textiles, such as clothes, especially underwear, sweatshirts, training suits and the like as well as towels etc., which are to have a good absorption property for sweat or other moisture. At the same time, especially for towels, the consumer desires that the textile be very soft. To achieve softness, soft rinsers, which are typically based on so-called “ester quats” and yield the desired softness, are added during the laundering process.

However, it is disadvantageous that such soft rinsers often have a hydrophobizing effect, i.e., reduce the capability for absorbing sweat, liquid or the like, and in addition cause a slightly oily feel which is uncomfortable to many consumers.

It is desirable to enable a textile treatment in which an improved adjustment between feel, softness and absorptivity or hydrophilicity/hydrophobicity is obtained. In particular, it is desirable to achieve improvements over existing soft rinsers in at least one of the aspects softness, feel and/or hydrophobizing property.

It is the object of the present invention to provide a novel textile treatment for industrial application.

This object is achieved by independent claim 1. Preferred embodiments are found in the dependent claims.

Thus, according to a first essential aspect of the invention, there is provided a textile treatment agent for the treatment of a textile to be contacted therewith, especially during a laundering process, having at least a first textile-treating fraction and at least one other fraction, wherein said first textile-treating fraction is designed to form an inorganic structure on the textile surface, especially the surfaces of the textile fibers.

The essential recognition is the fact that essential improvements can be achieved with an inorganic structure, to be applied to the fabric to be treated, which is invisible and typically cannot be felt itself. The non-feeling property is ensured if the layer is sufficiently thin, which is why layers of a thickness within a range of from 10 nm to 1 μm, preferably well under 1 μm, are preferred. It was found that the inorganic structure can substantially improve the water absorption rate of a textile and, depending of the thickness of the structure, also the water absorption quantity. The wearing comfort substantially increases, especially for textiles in which a high degree of sweat generation is to be typically expected, such as in sweatshirts etc., above all for fabrics of synthetic fibers. Also for towels, especially terry towels, the improved feel has a highly positive effect.

In a preferred embodiment, the textile-treating fraction contains or forms nanoparticles. Thus, inorganic structures with or of such nanoparticles form on the textile surface. If this structure containing or consisting of these nanoparticles is hydrophilic, the moisture can distribute over a larger surface area. In particular, this causes wet textiles to dry faster, which further increases the wearing comfort.

It may be noted that it is possible to inhibit bacterial and/or fungal growth by corresponding nanostructures. On the one hand, this can be promoted by nanostructures which have active components, such as SnO₂, ZnO, which are correspondingly bactericidal or fungicidal, and/or which additionally cause very fast drying; the faster drying is advantageous for suppressing the fungal growth because fission fungi typically thrive better on moister pieces of clothing.

The nanoparticles will typically be surface-modified, i.e., to be cationic, because textile fabric usually has a negative surface charge. It has been found that an inorganic structure can be built without any problems in the textile treatment with nanoparticles if the nanoparticles have a surface modification due to which the nanoparticles aggregate well with the fabrics having an essentially negative surface charge.

Typically, it is sufficient if the surface modification is performed with an amount of surface modification agent which is from 0.1 to 50%, based on the mass of the nanoparticles; preferably, it is provided for the surface modification agent to comprise from 1 to 20% of the nanoparticle mass. It may be clarified that the nanoparticles are first surface-modified, and then the textile is treated with those surface-modified nanoparticles to change their surface by the inorganic structure.

The surface modification may be organic or inorganic in nature. It is possible to use nanoparticles with both organic and inorganic surface modifications at the same time in the textile treatment agent.

The nanoparticles are preferably provided with a surface modified by Lewis acids. Oxides, hydroxides and/or salts may be provided. For cost reasons, aluminum chloride is particularly preferred, but it may be noted that additional effects can be obtained by selecting other substances.

It is possible to provide betains and/or silanes, especially organofunctional silanes, and/or cationic nanoparticles in the textile-treating fraction. These can be well controlled with respect to their chemical properties and are altogether suitable for the invention.

A further possibility is to provide substances in the first fraction which form nanostructures under application conditions, such as dilution with water and/or heating to temperatures typical of laundering processes (30° C., 60° C. or 95° C.).

As components forming nanoparticles, hydrolyzing salts can be provided, in particular, for example, aluminum chloride, TiOSO₄, ZrO₂ and/or silanes. Aluminum compounds are preferred merely for cost reasons and yield satisfactory results. The possibility to use so-called polymeric aluminum chloride or so-called polymeric aluminum oxide chloride as said nanoparticular substance or precursor thereof may be pointed out. It can be used both by itself and as a surface-modifying substance for nanoparticles, such as SiO₂ nanoparticles. This polymeric aluminum chloride or aluminum oxide chloride has a composition of AlClₓO_{y}, x typically being less than 3, and y typically being below 2, preferably above 0.1. This polymeric aluminum chloride or aluminum oxide chloride typically will still be water-soluble.

It is possible to provide a softener as another, especially second, fraction of the textile treatment agent, or to add components containing nanoparticles or inorganic structure-forming components to a soft rinser.

If desired, the textile treatment agent may be provided with detergents and/or care agents and/or perfumes in the usual way according to the market requirements, provided that the positive effect of the fraction according to the invention is not affected; rather, a cleansing during the application is even advantageous because it prepares the surface of a fabric for the structure formation in an optimum way without further measures being required.

The textile treatment agent can be applied to all kinds of textiles, such as those made of wool, cotton, silk, linen, microfibers, artificial fibers as well as mixed fabric.

For the textile treatment according to the invention, only relatively low amounts of material are required, typically about from 0.1% to 50%, preferably from 0.5% to 20%, based on the total mass of soft rinser substance or formulation.

The application and structure formation is simple; it is done during the laundering, and no particular aftertreatment of the washed textiles is required; rather, the treatment is fixed upon drying in the air, in a laundry-dryer and/or during the ironing of laundry which may still be wet.

It may be noted that the invention contributes to substantially improving the feel of the treated textile.

Just when aluminum chloride is employed, which is preferred, it is advantageous that no offensive smell is obtained, in contrast to acetates.

The invention will be described in the following by means of Examples:

A commercially available SiO₂ colloid dispersion with negative or neutral SiO₂, “Levasil 200S” in this case, is cautiously admixed with a maximum of 5% AlCl₃. This yields a surface modification with a positive surface charge of the SiO₂ substances contained therein.

The thus obtained intermediate product is stirred into commercially available soft rinser (Vernel “Pfirsich” in this case), namely in such an amount that a concentration of nanoparticles of 1.5% by weight results. The thus obtained preparation is added to cotton mixed fabrics and polyester washed at 60° C. with a heavy-duty detergent with usual machine laundering, followed by drying. Then, the water absorption rate is determined by a standardized TEGEWA test after the fabric has been ironed, and compared to fabrics rinsed with commercially available soft rinser. Thus, a drop of water is dropped onto the spanned textile from a defined height, and the penetration time is measured.

It is found that the soft rinser provided with nanoparticles has a water absorption rate for the different fabrics which is 10% to 20% higher. The feel of the two substances was evaluated by panelists. While conventional soft rinser obtained a rating of 3 on a scale from 1 (excellent) to 5 (deficient), the agent according to the invention obtained a rating of 1.3.

Subsequent repeated washes without the textile treatment agent according to the invention showed that the effect on the textile subsides without adverse effects on the fabric occurring. After one wash without the textile treatment agent according to the invention, an effect is hardly observable, and after two washes, it is no longer observable at all. Thus, the formed structure has been formed reversibly.

### EXAMPLE 2

10% AlCl₃ is added to Levasil 200S. The results are as above.

### EXAMPLE 3

Only almost 0.10/0 AlCl₃ is added to the Levasil. The results are worse, which is attributed to the fact that less of the substance is deposited on the fabric. This is again attributed to the fact that the surface of the Levasil was only slightly modified.

The effluent of the test series was examined, and only an at most low load with nanoparticles was established, which shows that an almost quantitative transfer to the fabric occurred. This is attributed to the fact that the nanoparticles modified to have a positive surface charge almost quantitatively arrive at the cleansed negative fiber surfaces. In addition, the substances remaining in the liquor can be rated as altogether harmless in the effluent.

1. A textile treatment agent having at least a first textile-treating fraction and at least one other fraction, characterized in that said first textile-treating fraction is designed to form an inorganic structure on the textile.

2. The textile treatment agent according to claim 1, characterized in that said first textile-treating fraction is present in an amount which is sufficient for building a layer having a thickness of about 10 nm to about 1 μm.

3. The textile treatment agent according to claim 2, characterized in that said textile-treating fraction contains nanoparticles.

4. The textile treatment agent according to claim 3, characterized in that said textile-treating fraction contains nanoparticles having a size of from about 5 to about 100 nm.

5. The textile treatment agent according to claim 4, characterized in that said nanoparticles are surface-modified.

6. The textile treatment agent according to claim 5, characterized in that the surface modification agent is present in an amount of between about 0.1% to 50% based on the nanoparticle mass.

7. The textile treatment agent according to claim 3, characterized in that said nanoparticles have an inorganic surface modification.

8. The textile treatment agent according to claim 7, characterized in that nanoparticles having surfaces modified by Lewis acids are provided.

9. The textile treatment agent according to claim 1, characterized in that oxides, hydroxides, salts, and combinations thereof are used for surface modification of the nanoparticles in the first fraction.

10. The textile treatment agent according to claim 9, characterized in that said first textile-treating fraction includes compounds for surface modification of the nanoparticles selected from the group consisting essentially of AlCl₃, ZrOCl₂, Ti and combinations thereof.

11. The textile treatment agent according to claim 1, characterized in that said first textile-treating fraction comprises nanoparticles having an organic surface modification.

12. The textile treatment agent according to claim 11, characterized in that substances selected from the group of betains and silanes, are provided for organic surface modification.

13. The textile treatment agent according to claim 1, characterized in that cationic nanoparticles are provided in said first fraction.

14. The textile treatment agent according to claim 1, characterized in that at least one component which forms nanostructures under application conditions is contained in said first textile-treating fraction.

15. The textile treatment agent according to claim 14, characterized in that said first textile-treating fraction includes hydrolyzing salts as said components forming nanostructures.

16. The textile treatment agent according to claim 1, characterized in that a softener is provided as a second fraction.

17. The textile treatment agent according to claim 1, characterized in that additional components are selected from the group, consisting of detergents curing agents and perfumes.

18. The textile treatment agent according to claim 1 for the treatment of a wool, cotton, silk, synthetic fiber or mixed fabric textile.

19. A soft rinser according to claim 1, characterized in that said first textile-treating fraction is provided in an amount of from 0.5 to 20%.

20. A method for treating textiles characterized in that an inorganic structure with nanosubstances is applied during washing and soft-rinsing, followed by fixing said inorganic structure with nanosubstances by drying and/or by ironing.
